(19) United States
(12) Patent Application Publication
(10) Pub. No.: US 2007/0015952 A1
(43) Pub. Date: Jan. 18, 2007

(54) Urinary frequency and urgency treatment device

(76) Inventors: T. Debuene Chang, Livermore, CA (US); Luiz B. Da Silva, Danville, CA (US)

Correspondence Address: John P. Wooldridge, 114 Honu'ea Pl., Kihei, HI, 96753, US

(21) Appl. No.: 11/434,616

(22) Filed: May 15, 2006

Related U.S. Application Data
(63) Continuation-in-part of application No. 10/673,916, filed on Sep. 26, 2003.
(60) Provisional application No. 60/681,185, filed on May 13, 2005.

(51) Int. Cl. A61F 2/00; A61H 1/00

(52) U.S. Cl. 600/29; 601/46

(57) ABSTRACT

A medical device is used to treat bladder urgency, frequency, and discomfort. The vibrational unit and power source can be contained together or separately. This device has external and intra-cavitary usage. For intra-cavitary usage, a means to retrieve the unit is provided.

This application claims priority to U.S. Provisional Patent Application Ser. No. 60/681,185, titled: “Urinary Frequency and Urgency Treatment Device”, filed May 13, 2005, incorporated herein by reference.

This is a continuation-in-part of U.S. patent application Ser. No. 10/673,916, titled: “Urinary Diagnostic System Having a Retrievable Sensing Device”, filed Sep. 26, 2003, incorporated herein by reference.

### BACKGROUND OF THE INVENTION

1. Field of the Invention

The present invention relates to medical devices for the treatment of pain and more specifically, it relates to a system that can be used to treat bladder urgency, frequency and pain.

2. Description of Related Art

Abnormal urinary voiding patterns are very common worldwide, affecting both women and men. For incontinence alone, the World Health Organization estimates that there are 200 million men and women worldwide with symptoms of urinary incontinence. This condition affects the patient both physically and emotionally—affecting the patient's quality of life. It is predicted that urinary incontinence will affect an estimated 30 million American women of all ages. This condition can interfere with work, travel, recreation and sexual activities. It is also associated with urinary tract infections and scrotal, perineal, and labial ulcers. Male incontinence is most commonly caused by prostatic enlargement and/or surgery. Female incontinence often results from stretching of the pelvic support structures during pregnancy and childbirth. Post menopausal women as well as those who have undergone a hysterectomy are additional contributory factors. In both sexes, other contributing factors to urinary incontinence include obesity, cigarette smoking, pelvic radiation therapy, diabetes, Parkinson's disease, back injury, cerebral vascular accident, and dementia.

There are a variety of treatment options for bladder control problems ranging from behavioral therapy, pelvic muscle exercises, biofeedback, bladder training, fluid/dietary modifications, medications, urethral devices, pelvic floor supporters, and surgical procedures such as bladder suspensions, slings, and urethral bulking procedures. In relatively minor cases, satisfactory treatment may include the use of absorbent pads combined with strengthening exercises and behavior modification. Anti-spasmodic medications may be added to the multi-modality regimen for treatment. Surgery may become necessary if these combinations of non-invasive treatments are inadequate. Various numbers of surgical options are currently available with many surgeries reinforcing and supporting the bladder neck region, either with anterior suture support or posterior anchored suture or sling support. Other procedures available include surgical injections of urethral bulking materials or elements that mechanically close the urethral opening (e.g., an inflatable balloon; a clamp).

A surgical procedure, often reserved for intractable pain and urgency, is surgical implantation of a high frequency device to stimulate the sacral nerves. (Medtronics-Interstim, Minneapolis, Minn.). The theory behind the stimulation of the sacral nerves is that this high frequency device will suppress the reflexive arch of the pelvic nerves, thereby decreasing the sensation of urgency and frequency. In addition, the afferent pathways for muscle stretch receptors and fine touch proprioception travel briefly together with the vibration receptors in the dorsal column of the spinal column on the way to the pontine regions of the brainstem.

Many female patients with urinary frequency and incontinence have loss of normal support of the pelvic floor. Mechanical movement of the bladder neck occurs when the pelvic support floor is lost. This is apparent from both physical exam and radiological studies correlated with video urodynamics. Theoretically, in a similar manner, exaggerating the increased motion of the bladder and bladder neck with repetitive, high frequency vibrational stimulation of bladder stretch receptors might dampen the sensation for frequency and urgency by overstimulating the receptors and raising the inherent threshold for the signal for voiding. This can be accomplished with either internal or external methods with mechanically or acoustically generated vibrations. More evidence for an alternative etiology of bladder frequency and urgency with loss of the pelvic floor comes from urodynamic studies. Pressure studies of these patient's bladders during filling often fail to show any abnormalities although some patients show small, minor contractions during normal filling (uninhibited contractions). Compliance in most of these patients during filling is normal and does not increase until the bladder contracts to void. Pressure increase alone is not the reason that patients develop frequency and urgency when they lose pelvic floor support as bladder pressures remain quite normal in most patients. The loss of the pelvic floor support apparently changes the threshold for voiding to the bladder and results in the sense of urgency and frequency. What is not clear is why movement of the bladder and bladder neck changes the signals for voiding.

Other urinary disorders that apparently alter the threshold for voiding include urinary tract infections and benign prostatic hypertrophy in men. In urinary tract infections, urgency and frequency is a common symptom. Cystoscopic examination shows edema and inflammation. The bladder remains unchanged in size but is unable to hold more urine during the infection as the sensation to void forces patients to empty their bladder frequently. In benign prostatic hypertrophy, the bladder works against an obstruction of the prostate. Nocturia and frequency are common symptoms.

Current therapies to frequency and urgency are varied and depend on the etiology. In urinary tract infections, multiple antibiotics are available for treatment. Unfortunately, symptoms from urinary tract infections last for many days after the infection is actually treated. There are few therapies for patients during the time necessary for the edema and erythema from the urinary tract infection to abate. For intractable urgency and frequency, some current treatment modalities are invasive and include surgical treatment and implantation of devices. There is need for a non-invasive device to treat and augment current therapies for urinary frequency and urgency.

For patients with urinary frequency and urgency from loss of the pelvic support structures, treatment modalities are more limited. Some treatments are (i) strengthening the pelvic muscles with Kegel's exercises, (ii) biofeedback, (iii) pessaries and (iv) vaginal exercise equipment. Pharmacological treatment to help relax the bladder and tighten the opening are available but require patients to be on the medications long term and have a multitude of major side effects. Many patients become non-compliant with the anticholinergics (the predominant form of pharmacological treatment for frequency and urgency) either because of the side effects or because patients do not subjectively feel significant improvement in their lifestyles. Moreover, the effects of many of these medications last for days and may severely affect the elderly. More invasive treatments for the loss of support of the pelvic floor include surgical interventions with reinforcement of the pelvic floor.

A non-invasive, easily placed and removable device to treat urinary frequency is needed and would greatly improve the options for treatment. An effect device usable for short periods of time would be useful for treatment of the frequency and urgency of patients who are treated for urinary tract infections but are still symptomatic. Such device should give temporary relief for urgency, bladder discomfort, and lower abdominal pain of chronic cystitis. In patients with urinary frequency and urgency from loss of the pelvic support structures, there is a need for a non-invasive device that will give relief. In patients with urge incontinence from benign prostatic hypertrophy, there is a need for a non-invasive device that will allow more holding time to prevent incontinence. Given the limitations of current techniques to non-invasively treat both short and long term urinary frequency and urgency, there is a need for a novel device and system that can simplify and improve treatment of urinary frequency and urgency. The present invention fulfills these needs, and further provides related advantages.

### SUMMARY OF THE INVENTION

An object of the present invention is to provide a non invasive and miminally invasive device and method to treat bladder frequency, urgency, pain and other bladder abnormalities.

It is another object to provide a vibrational stimulation device and method for pelvic treatment.

These and other objects will be apparent to those skilled in the art based on the teachings herein.

A non-invasive element is placed on the skin, intrarectally, or intravaginally to treat continuous or intermittant urinary urgency and frequency. The device may be activated and deactivated by a bladder sensor, voice or remote controlled activated, or automatic timed activation. The present invention provides the physician with an alternative treatment modality for patients that is non-invasive or minimally invasive and has minimal side effects. Furthermore, the treatment modality is easily reversible once the need is resolved.

Treatment for bladder frequency and urgency and pain by a vibrational device is novel. Currently, there are no effective devices with similar modalities for the treatment of urinary frequency and urgency. Further, there are few effective treatment devices for urinary frequency and urgency which are non invasive, have minimal side effects, and are easily implemented, reversible and removable. Unlike a surgically implanted unit which can be difficult to remove, the present device may be applied externally to the abdominal skin or placed in the vagina or rectum and can easily be removed with minimal disruption in a patient's normal day. The device can be tolerated for use over many hours or days.

Because of the ease of application and removal, patients can use this unit for both temporary and long term use. Unlike surgical procedures, this unit is practical for short term use. One ideal use is for patients who have been treated for urinary tract infections with effective antibiotics but who still have symptoms or who are expected to have symptoms for days after the acute infection has been treated. Use of this unit will effectively return the patient to a productive quality of life at an earlier time. Because of this, there is wide applicability of this unit in a significant portion of the population of all sexes and ages. Long term use in patients who either choose not to have surgical intervention or are not candidates for surgical intervention will significantly improve their quality of life with minimal risk. There is wide applicability of this device for treatment of urinary frequency and urgency and such treatment will significantly improve a patient's quality of life.

In the intravaginal embodiments of the current device, another benefit of a properly placed device is a mechanical support of the pelvic floor, much like a pessary. Unlike conventional pessaries, embodiments of the present device act to suppress bladder contractions, urgency, and frequency.

Currently, pharmacological control of urinary frequency and urgency is commonly used. Because pharmacological agents are systemic in application, side effects from the medications are rate limiting for many patients to obtain effective therapy. This is especially true for the elderly of either sex. The major complaint which precludes patients from continuing therapy with the anticholinergics is dry mouth. Because of the systemic effects of medications, patients with concurrent disease processes such as narrow angle glaucoma and benign prostatic hypertrophy are precluded from taking the majority of these medications. One major advantage of the present devices is their localized application without systemic effect. Because of this, the present devices are safer to use for a much larger segment of the population, regardless of age or sex.

### BRIEF DESCRIPTION OF THE DRAWINGS

The accompanying drawings, which are incorporated into and form part of this disclosure, illustrate embodiments of the invention and together with the description, serve to explain the principles of the invention.

FIG. 1 shows an embodiment of the device in which the vibrating element connects to a separate control unit and power supply.

FIG. 2 shows an embodiment of the device in which the device is a single unit which integrates vibrating element, power supply, and control eletronics.

FIG. 3A shows use of alternative intra-cavitary embodiment of the vibrating device with retrieval mechanism.

FIG. 3B shows the device of FIG. 3A.

FIG. 4A shows use of an alternative intra-cavitary embodiment of the device.

FIG. 4B shows the device of FIG. 4A.

FIG. 5A shows use of an alternative intra-cavitary embodiment of the device.

FIG. 5B shows the device of FIG. 5A.

FIG. 6A shows use of an alternative intra-avitary embodiment of the device.

FIG. 7 shows a view of a vibrational unit

FIG. 8 shows a view of an external control box.

### DETAILED DESCRIPTION OF THE INVENTION

One embodiment is in two parts, a small, disposable patch adherent to the skin on the lower abdomen or suprapubic region connected to a power source and control unit that e.g., may be worn on the belt and attached by a thin wire. The patch and/or the power source can be changed as necessary. The patch system provides a source of vibrations to suppress and dampen the contractions of the detrusor muscle of the bladder and can be easily implemented regardless of age or sex. Vibrations are generated by an unbalanced shaft motor that is contained within the patch. The vibrations are in the range of 100 Hz to 10 kHz. The patch is attached with biocompatible adhesive and contained within biocompatible materials and placed on the mid lower abdomen for proper positioning over the bladder. It can be covered by waterproof, biocompatible material if desired. Properties of the biocompatible material would be minimal to no reaction with skin, low to no toxicity, and will durable for the length of the use of the device which may be about 1-2 months. The power source and control unit are contained in a separate unit, approximately 7.5 cm×6 cm×2 cm in size. The power source (e.g., a battery) can be changed if necessary by opening up the control unit or can have an electrical plug for recharging. The control unit is linked by a flexible wire to the patch. The control unit can be embedded in specialized underwear or can be attached to a belt to carry and support the unit in position. The control unit will contain a computer chip and can be pre-programmed by the physician or adjusted by the user with a variety of options to run the vibrational patch device. These option for the control unit can include a physical on/off button, a voice activated mechanism to recognize specific words by the user or generic claps or snaps and other sounds. Other control options can include a hand-held remote control unit to provide a signal or a remotely controlled bladder sensor which sends a signal when a bladder contraction or pressure increases in the bladder to the control unit. See U.S. patent application Ser. No. 10/673,916, incorporated herein by reference. These signals from the bladder sensor would be received by the control unit which will then automatically turn on the vibrational patch to start vibrations and suppress the bladder contraction.

In another embodiment, a self contained unit with the vibrational unit and power source can be positioned over the lower abdomen by a belt or embedded into underwear. This unit is covered with a biocompatible material and can be made waterproof. Adhesion of this self contained unit can be through direct contact with the skin with a biocompatible adhesive or embedded in specialized underwear. Again, biocompatible materials need to have properties of minimal to no skin reaction, little to no toxicity, and enough durability for the length of use of the units about 1-2 months. Like the first embodiment, adjustments are similar with, e.g., an on/off button, device preprogramming, voice activation, or remote control with either an external device or feedback from a bladder sensor. The self contained unit would contain all the other elements of the first embodiment in one unit with an unbalanced shaft motor to generate vibrations in the range of 100 Hz-10 kHz, a power source which can be changed by opening up one side of the device, and a receiver or pre-programmed chip for controlling the on/off position of the unit. A physical on/off button can be included on this unit for user control.

In another embodiment, made specifically for intravaginal or intrarectal placement, a retrievable self contained unit is configured for placement entirely in the vagina or rectum, and includes an attached flexible string. The intravaginal device is approximately 15 cm length×3 cm diameter at its widest point at mid shaft. The intrarectal device is smaller at 8-10 cm length×1.5 cm diameter at the widest point at mid shaft. Everything else about the units are similar. Each unit is entirely self contained. It is covered in biocompatible material which has no toxicity in the vaginal wall or colonic/rectal mucosa, is not reactive to mucosa, and can be waterproof to protect the internal components. The device includes a power source contained entirely in the unit. The unit is designed to be removed from time-to-time for cleaning and for changing the power source. The unit is retrieved by a retrieval string attached at the distal end of the oblong shaped device. This unit produces vibrations of 100 Hz to 10 kHz, generated by an unbalanced motor to suppress urgency and frequency. The unit has a completely smooth surface with no sharp regions. It includes a long and oblong shape and is firm but smooth for finger placement into position. This is essential for placement and removal of the units in the delicate mucosa tissue. Because of the internal placement of these units, no controls will be available for the user on the outside of the device. All controls are either preprogrammed in computer chip within the unit or from outside remote control. The device includes a receiver unit to receive these outside remote signals. Signals can be from a variety of wireless technology sources including Bluetooth transmission, radiofrequency, AM or FM. Voice activation is possible in this device as well as an outside remote control, much like a TV remote control or a garage opener. A biofeedback from a bladder sensor, contained within the bladder, which generates signals to the vibrational device when a high pressure is generated in the bladder, is one possibility of control for this device. The bladder sensory control unit is floated in the bladder. It transmits by radiofrequency or other wireless modality when there is an increase of bladder pressure to a vibrational unit. The vibrational unit includes a receiving unit built in with a preprogrammed time and frequency to operate whenever an increased pressure is detected by the bladder sensor. This is for the internal devices. For the external devices, a similar process is possible or an external on/off switch or lever may be started by the patient. An external device with a self operated on/off switch would not need a bladder sensor and will rely on the patient's own sensation.

An embodiment of the device can be contained in a pessary of different shapes, which is placed intravaginally. Different pessary shapes are available. One of the common shapes is a donut like shape that fits under the pubic bone within the vagina. Like the other internal devices, this device includes a biocompatible covering which is non reactive, non toxic, durable and waterproof. It has smooth surface and is removable for cleaning. The power source is changeable or can be recharged. The unit is self contained like the previous embodiment and will contain all the same elements of the unbalanced shaft motor to generate vibrations of the range of 100 Hz to 10 kHz, a programmable chip or a transmission receiver, depending on the mode of control of the device. This unit can be controlled by an outside remote control, preprogrammed chip, or biofeedback from a bladder sensor for increased bladder pressure. Removal of this device is by finger manipulation to extract the device for cleaning and changing or recharging the power source. The vibrational generation can be done in 2 different forms in this device. In one form, a media which transmits acoustic waves is used to fill the donut shaped portion circumferentially to evenly transmit the vibrations. The unbalanced shaft motor is contained at one end of the unit but because of the media, it will generate waves circumferentially to the whole ring. In another embodiment, the vibrational unit is located in the anterior region of a donut portion. This transmits vibrations locally to the base of the bladder, the site of the trigone, which is most sensitive for bladder control. In this embodiment, the pessary shape of the device in the vagina serves more than one function as pessarys are used to help secure the pelvic organs in proper position.

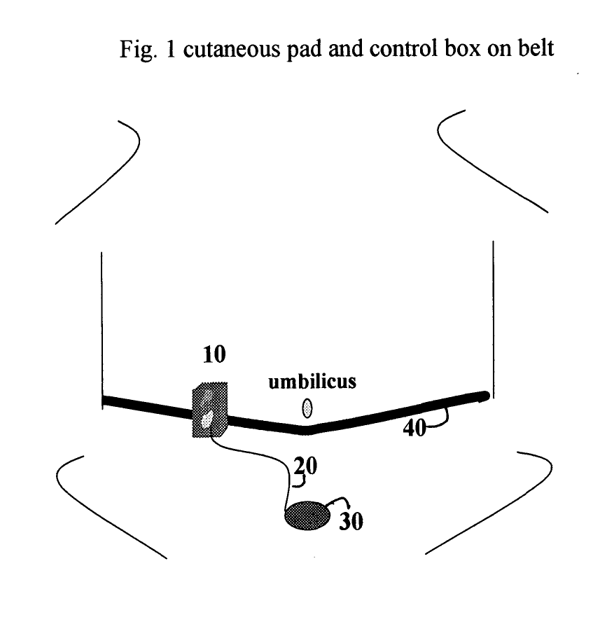

FIG. 1 depicts a body sketch of a lower abdominal wall and pelvis with application of an embodiment of the system. Unit 10 is the control unit and contains the power source, control electronics, including e.g., hardware and software, on/off switch and may include one or more remote sensing elements. Unit 10, the control unit, is attached to the patch which contains the vibrational unit by wire 20. The vibrational unit is part of a disposable patch 30 for placement on the skin. Unit 10 is held on the body by a belt 40 in this embodiment. Alternately, belt 40 may be eliminated by embedding unit 10 into specialized underwear.

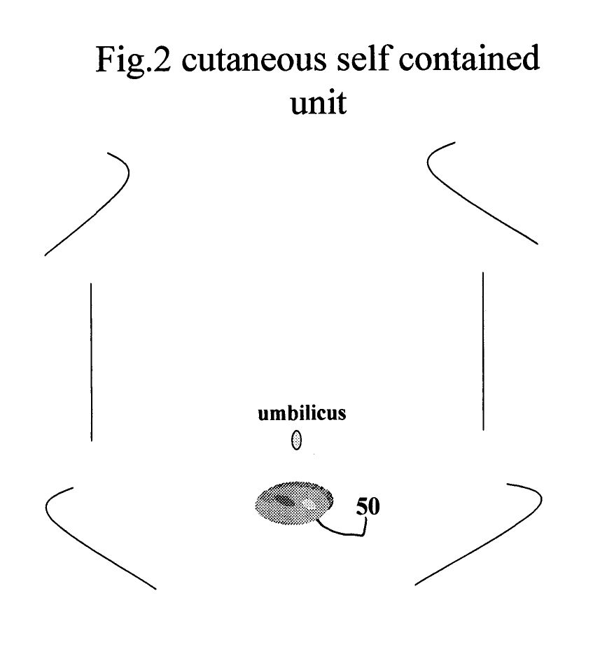

FIG. 2 illustrates another embodiment of the system and how it would be used in accordance with the present invention. Unit 50 is self contained and is attached to the skin surface with disposable adhesive. Unit 50 includes a power source (e.g., battery), the vibrating element which is a rotating motor with an unbalanced shaft, and control electronics. This unit can include a receiver that allows remote control.

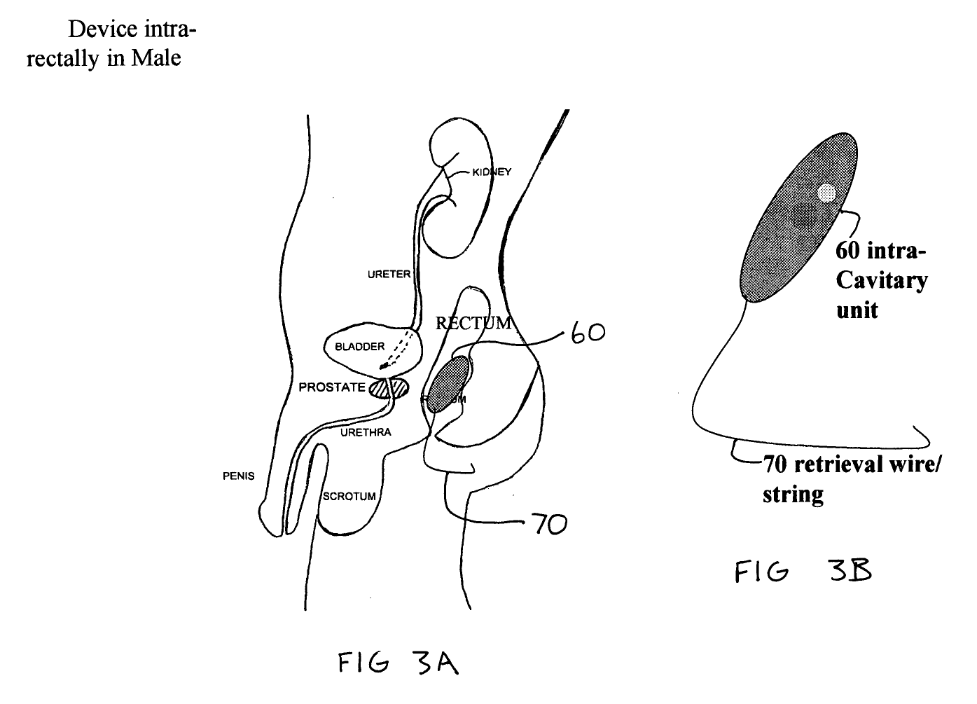

FIG. 3A illustrates use of an embodiment of an intracavity unit 60 in position in the rectum in a male pelvis with retrieval wire/string 70. FIG. 3B shows the device of FIG. 3A. This unit is self contained with power source, remote receiver, vibrating element, and control electronics integrated within unit. Placement is by insertion from the blunt end of the unit 60 and retrieval is with pulling on the wire/string 70.

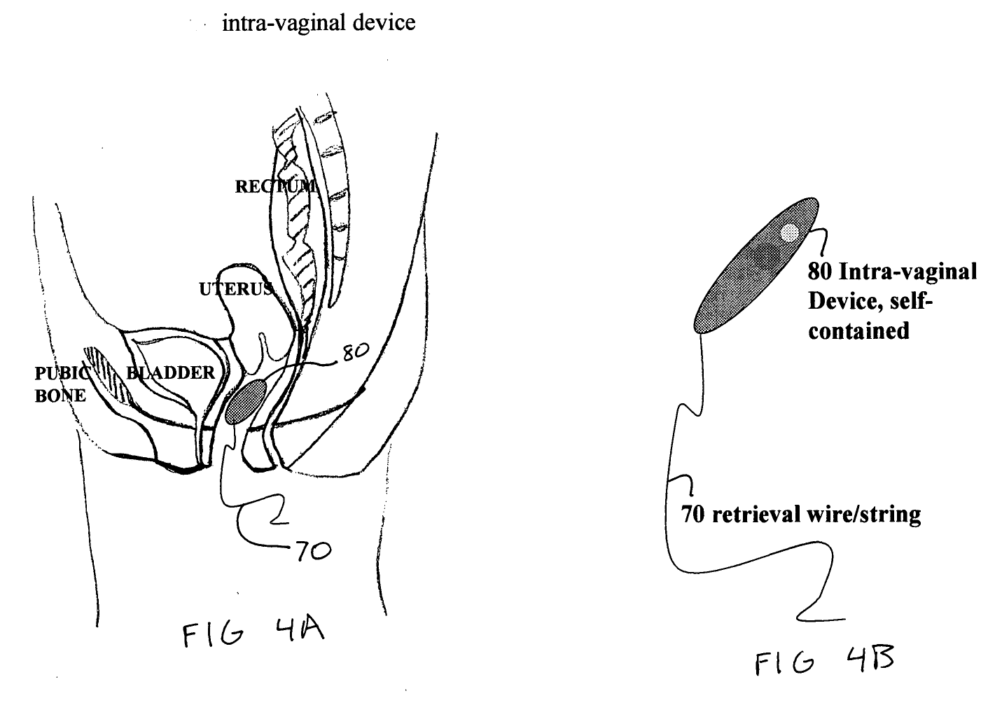

FIG. 4A shows us of another embodiment for placement in the vagina. FIG. 4B shows the device of FIG. 4A. Unit 80 is self contained and placed with pushing in the direction of the longitudinal axis of the vagina by blunt pressure on the end of the unit. Retrieval is with pulling of the string/wire 70. This unit is self contained with power source, remote receiver, vibrating element, and control electronics integrated within unit 80.

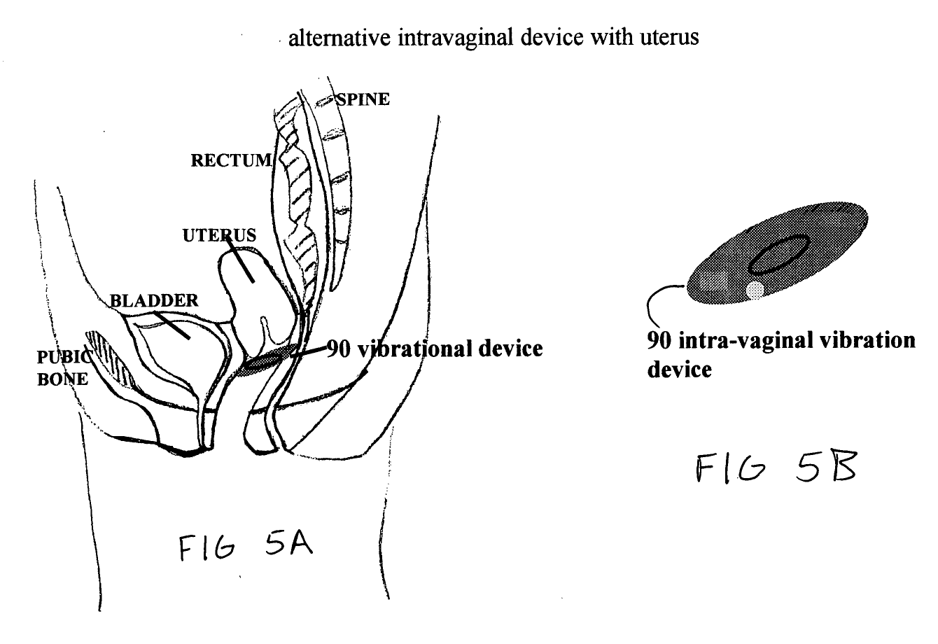

FIG. 5A shows use of a proximal vaginal embodiment of the device 90. Within the unit, there is a vibrational unit, viscous matrix to transmit the vibrations to the surface of the device, power source, remote receiver, and control electronics. FIG. 5B shows the device of FIG. 5A. The unit is flexible and placement is by folding the donut for insertion into the vagina. Removal is by finger placement through the hole in the donut and hooking the finger around a wedge of the donut. Shown is the device in place in female pelvis with uterus present

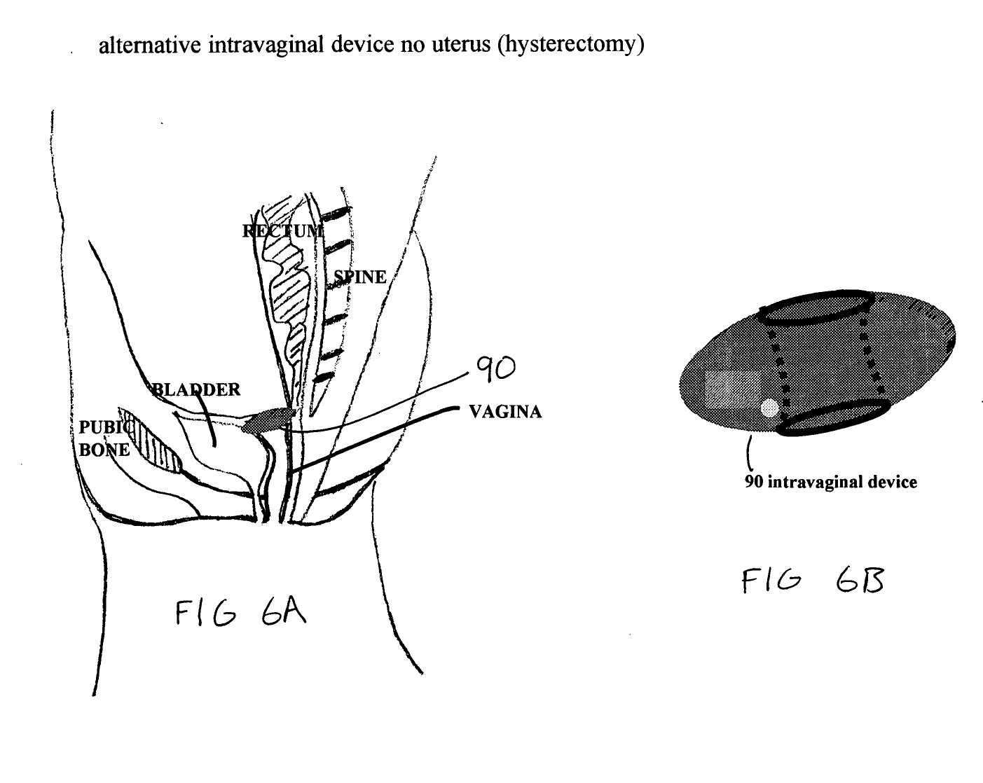

FIG. 6A shows use of an embodiment of the device 90 in a female pelvis that has had removal of uterus (hysterectomy). FIG. 6B shows the device of FIG. 6A. The device is round donut shaped, soft and pliable. It is hollow in the center and placed by folding in half and inserting the leading edge into the vagina proximally. Removal is by hooking a finger into the center of the donut and pulling on an edge of the donut shaped device. This unit is self contained with power source, remote receiver, vibrating element, and control electronics integrated within unit

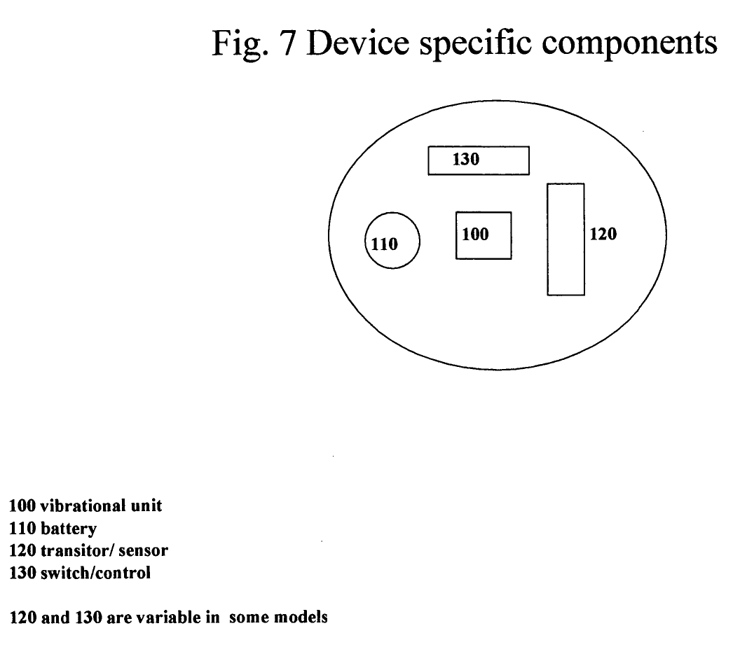

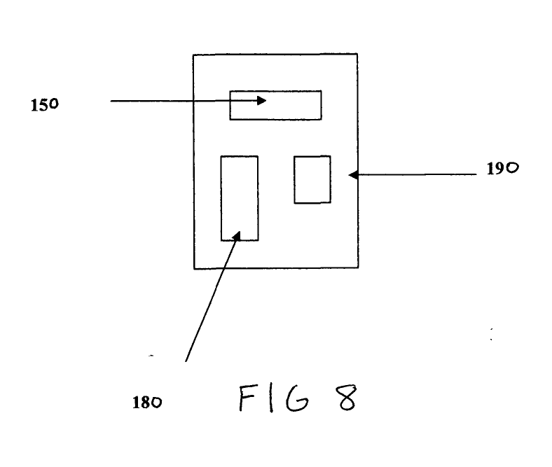

FIG. 7 shows components of a self contained embodiment, including vibrational unit 100, battery 110, transistor/sensor 120, and switch/control 130. FIG. 8 illustrates elements of an external control box, for use with units that are not self contained. It includes a control switch 150, a battery 180 and a receiver/transistor/program chip 190.

In all embodiments of the device the vibrating element vibrates at a fixed frequency, a user selected frequency, or over a frequency range. In most cases the vibration frequency will be less than 10 kHz and usually in the range of 1 Hz to 5 kHz. The vibrating element can use a mechanical method such as a miniature motor with an off balance rotor. Alternatively the vibration could be produced by piezoelectric, or electromagnetic devices.

The foregoing description of preferred embodiments of the invention is presented for purposes of illustration and description and is not intended to be exhaustive or to limit the invention to the precise form disclosed. Many modifications and variations are possible in light of the above teaching. The embodiments were chosen and described to best explain the principles of the invention and its practical application to thereby enable others skilled in the art to best use the invention in various embodiments and with various modifications suited to the particular use contemplated.

We claim:

1. A method for treating bladder frequency and urgency, comprising:
contacting a vibrator to a location on or in the body of a patient under treatment, wherein said location is selected from the group consisting of the dermis, inside the rectal cavity and inside the anal cavity; and
transmitting vibrations from said vibrator to the detrusor muscle of the bladder of the patient.

2. The method of claim 1, wherein said vibrations comprise a vibration frequency that is less than 10 kHz.

3. The method of claim 1, wherein said vibrations comprise a vibration frequency within a range from 1 Hz to 5 kHz.

4. The method of claim 1, wherein said vibrator comprises a miniature motor with an off balance rotor.

5. The method of claim 1, wherein said vibrations are produced by a piezoelectric device or an electromagnetic device.

6. The method of claim 1, wherein said vibrator comprises:
a vibrating element;
a power source operatively connected to provide power to said vibrating element;
control electronics operatively connected to control said vibrating element; and
means for attaching or placing said vibrating element in contact with said location.

7. The method of claim 6, wherein said control electronics include a receiver operatively connected to said vibrating element, wherein said receiver is configured to accept control commands

8. The method of claim 6, wherein said vibrating element is integrated into a disposable patch that includes adhesive.

9. The method of claim 6, wherein said vibrating element is integrated into a piece of cloth.

10. The method of claim 6, wherein said control electronics include a preprogrammed microchip that controls at least one parameter of said vibrating element.

11. The method of claim 6, further comprising operatively positioning a bladder sensor configured to collect data indicative of the extent to which the bladder is full, wherein said control electronics include means for reading said data and controlling at least one parameter of said vibrating element in response to said data.

12. The method of claim 11, wherein said means for reading said data and controlling at least one parameter of said vibrating element is wireless between said bladder sensor and said control electronics.

13. The method of claim 6, wherein said control electronics are voice activated.

14. The method of claim 6, wherein said vibrating element, said power source and said control electronics are contained within a flexible housing.

15. An apparatus for treating bladder frequency and urgency, comprising:
a vibrating element;
a power source operatively connected to provide power to said vibrating element;
control electronics operatively connected to control said vibrating element; and
means for attaching or placing said vibrating element device in contact with a location selected from the group consisting of the dermis, inside the rectal cavity and inside the anal cavity, wherein said vibrating element is configured to transmit vibrations from said vibrator to the detrusor muscle of the bladder of the patient.

16. The apparatus of claim 15, further comprising a bladder sensor configured to collect data indicative of the extent to which the bladder is full, wherein said control electronics include means for reading said data and controlling at least one parameter of said vibrating element in response to said data.

17. The apparatus of claim 15, wherein said means for reading said data and controlling at least one parameter of said vibrating element is wireless between said bladder sensor and said control electronics.

18. The apparatus of claim 16, wherein said bladder sensor comprises:
a retrieval wire configured to extend from the interior of a bladder, wherein said bladder sensor is configured to collect data;
a deployable housing, said housing having a body defining an interior and an exterior, said housing configured to allow fluid to flow through said housing, wherein said sensing device is disposed within said interior of said housing;
a disposer for disposing said housing having said sensing device into said body part, wherein said deployable housing expands when disposed into said body part to prevent ejection of said sensing device from said body part.

19. The apparatus of claim 18, wherein said bladder sensor is selected from the group consisting of pressure sensing devices, thermal sensing devices, conductive sensing devices, capacitive sensing devices, inductive sensing devices, resistive sensing devices, and optical pressure sensing devices.

20. The apparatus of claim 18, wherein said retrieval wire is configured to operate as an antenna.
